(19) United States
(12) Patent Application Publication
(10) Pub. No.: US 2023/0354790 A1
(43) Pub. Date: Nov. 9, 2023

(54) STERILE ORGANISMS, METHODS OF MAKING, AND METHODS OF USE THEREOF

(71) Applicant: Brandeis University, Waltham, MA (US)

(72) Inventors: Paul Allen Garrity, Newton, MA (US); Willem Jeffrey Laursen, Waltham, MA (US)

(21) Appl. No.: 18/246,250

(22) PCT Filed: Sep. 28, 2021
(86) PCT No.: PCT/US2021/052374

§ 371 Date: Mar. 22, 2023

Related U.S. Application Data
(60) Provisional application No. 63/084,683, filed on Sep. 29, 2020.

(51) Int. Cl. A01K 67/033; C12N 9/22; C12N 15/11; C12N 15/90; C07K 14/435

(52) U.S. Cl. CPC A01K 67/0339; C12N 9/22; C12N 15/111; C12N 15/902; C07K 14/43563; C12N 2310/20; C12N 2800/80; A01K 2227/706; A01K 2217/072; A01K 2267/02

(57) ABSTRACT

A method of making sterile diploid organisms includes mating a first population and a second population of single knock-in diploid organisms, wherein the first population of single knock-in diploid organisms are heterozygous organisms expressing a first marker inserted into a gene required for fertility, wherein the second population of single knock-in diploid organisms are heterozygous organisms expressing a second marker inserted into the gene required for fertility, wherein introduction of the first/second marker disrupts expression of the required fertility gene creating a first/second mutant allele of the gene required for fertility, and wherein the first and second markers are distinct; sorting offspring produced from the mating based on their expression of the first and/or second markers; and isolating the sterile diploid organisms, wherein the sterile diploid organisms are heteroallelic diploid organisms expressing the first marker in the first mutant allele and the second marker in the mutant second allele.

### CROSS-REFERENCE TO RELATED APPLICATIONS

This application claims priority to U.S. Provisional Application 63/084,683 filed on Sep. 29, 2020, which is incorporated herein by reference in its entirety.

### BACKGROUND

“Sterile Insect Technology” (SIT) is a powerful and environmentally friendly strategy for mitigating and even eradicating insect pests and vectors of disease. In SIT, sterile male insects of a given species are released into the environment to compete with their wild male counterparts for mating to wild females. Mating to sterile males leads to species-specific reductions in the levels of reproduction followed by declines in population size, in some cases driving the population to zero. A major bottleneck in implementing SIT against many species is the difficulty and expense in generating large numbers of males that reliably fail to produce viable offspring but are otherwise fit and effective at mating.

Sterile males can be generated in a variety of ways, from irradiation to the introduction of a sterilizing pathogen or transgene. However, these strategies commonly suffer from either causing additional damage that harms mating fitness, requiring substantial optimization (limiting applicability to new species) or resulting in the introduction of factors (pathogens) that can have broader ecological impacts.

What is needed are simple and generalizable molecular genetic strategies for generating males for use in SIT.

### BRIEF SUMMARY

In an aspect, a method of making sterile diploid organisms comprises mating a first population of single knock-in diploid organisms and a second population of single knock-in diploid organisms, wherein the first population of single knock-in diploid organisms are heterozygous organisms expressing a first marker inserted into a gene required for fertility, wherein introduction of the first marker disrupts the function of the gene required for fertility creating a first mutant allele of the gene required for fertility, wherein the second population of single knock-in diploid organisms are heterozygous organisms expressing a second marker inserted into the gene required for fertility, wherein introduction of the second marker disrupts expression of the gene required for fertility creating a second mutant allele of the gene required for fertility, and wherein the first and second markers are distinct; sorting offspring produced from the mating based on their expression of the first and/or second markers; and isolating the sterile diploid organisms, wherein the sterile diploid organisms are heteroallelic diploid organisms expressing the first marker in the first mutant allele and the second marker in the mutant second allele of the gene required for fertility.

In another aspect, a sterile diploid organism is produced by the foregoing method.

In yet another aspect, a method of mitigating or eradicating arthropods in an arthropod population comprises releasing into the arthropod population sterile arthropods produced by the foregoing method.

In a further aspect, a method of mitigating or eradicating arthropods in an arthropod population comprises releasing into the arthropod population a population of heteroallelic sterile arthropods expressing first and second markers; wherein introduction of the first marker disrupts function of a gene required for fertility to create a first mutant allele of the gene required for fertility, and introduction of the second marker disrupts function of the gene required for fertility to create a second mutant allele of the gene required for fertility; and wherein the first and second markers are different.

### BRIEF DESCRIPTION OF THE DRAWINGS

FIG. 1 shows knock-ins to the IR93a gene identical except for their fluorescent label, produced in RFP and EYFP versions. RFP/YFP co-expression indicates both the RFP-linked knock-in allele and the EYFP-linked knock-in allele are present and also indicates that the animal does not include a wild type allele.

FIG. 2a-c show A. gambiae mosquitoes carrying different Ir93a allele combinations. FIG. 2a shows larvae, 2b shows pupae and 2c shows adults. Ir93a alleles are: wild type (Ir93a^{wt}), an RFP insertion that knocks out Ir93a function (Ir93a^{RFP}), and a EYFP insertion that knocks out Ir93a function (Ir93a^{YFP}). Animals containing two wild type Ir93a alleles are not fluorescent Animals containing one Ir93a^{RFP} and one Ir93a^{YFP} allele (Ir93a^{RFP}/Ir93a^{YFP}) express both RFP and EYFP and lack Ir93a function Animals expressing only one XFP (e.g., RFP) contain either one mutant and one wild type allele (e.g., Ir93a^{RFP}IIr93a^{wt}) or two mutant alleles of the same color (Ir93a^{RFP}/Ir93a^{RFP}). Ir93a* denotes this ambiguity. Note that this ambiguity does not compromise the utility of the PCK approach. Rather, the ambiguity associated with reliance on a single marker is why the PCK approach is powerful: it unambiguously identifies mutant animals.)

FIGS. 3a and b show experiments performed in D. melanogaster. 3a shows D. melanogaster fruit flies carrying different zpg allele combinations. zpg alleles are: zpg^{wt}, wild type allele; zpg^{RFP}, an RFP insertion that knocks out zpg function; and zpg^{YFP}, a YPF insertion that knocks out zpg function. Animals containing two wild type zpg alleles are not fluorescent Animals containing one zpg^{RFP} and one zpg^{YFP} allele (zpg^{RFP}/zpg^{YFP}) express both RFP and EYFP and lack zpg function Animals expressing only one XFP (e.g., RFP) contain either one mutant and one wild type allele (e.g., zpg^{RFP} zpg^{wt}) or two mutant alleles of the same color (e.g., zpg^{RFP} zpg^{RFP}). zpg* denotes this ambiguity. 3b shows matings between wild type males and females yield progeny. Progeny are visible as larvae and pupae on vial walls. Mating crosses between zpg mutant males and wild type females fail to yield progeny as do crosses between zpg mutant females and wild type males.

The above-described and other features will be appreciated and understood by those skilled in the art from the following detailed description, drawings, and appended claims.

### DETAILED DESCRIPTION

Described herein are novel methods of making sterile diploid organisms, specifically arthropods, and the sterile organisms produced by the methods. The method described herein is referred to as PCK-based genetic sterilization. Also included are methods of arthropod control using the sterile arthropods described herein.

Although a recessive sterile mutation can be propagated through reliance on fertile heterozygous animals, it is important to consider what the offspring of these heterozygotes will be genetically. When one creates a population of heterozygotes, the patterns of Mendelian inheritance mean that the offspring of such a stock will fall into three classes: homozygous wild type (+/+), heterozygous mutant (+/−) and homozygous mutant (−/−). From among these progeny, one can obtain homozygous mutants which can be used for SIT as well as heterozygous mutants which can be subsequently mated to produce the next generation. However, the challenge is to cheaply, efficiently and noninvasively distinguish these different genetic classes of animals from one another as well as from homozygous wild type animals (tracking homozygous wild type animals is useful as, if they are not discarded, they could eventually come to dominate the stock, crowding out the desired heterozygous mutant animals) An important point is that most sterility-inducing mutations do not result in a homozygous phenotype that can be easily detected by methods amenable to large-scale sorting. Thus, while these three classes of progeny can be generated, telling them apart on a large scale is a major challenge. In Drosophila melanogaster, this challenge is addressed through the use of complicated, marked “balancer” chromosomes. Importantly, balancer chromosomes are only useful in their species of origin, so the process would need to be repeated for each species where one wants to use them. However, developing balancer chromosomes required decades of effort in Drosophila and would not be simple to reproduce in other species. Due to the many pest species and vectors (and subpopulations thereof), SIT ideally requires the use of an alternative strategy for working with sterile mutants that can be readily ported to a new species or population.

To address the challenge of distinguishing among different classes of progeny without balancer chromosomes, a simple genetic strategy in which markers, e.g., fluorescent markers, are knocked-in to the gene(s) of interest to generate labeled mutations is described herein. While the knock-in of a fluorescent marker is well-established, the key difference in the strategy described herein is that rather than making a single knock-in mutation allele, two knock-in alleles were created that are essentially identical at the molecular level (the endogenous gene is disrupted at the precisely same site), except that each harbors a different fluorescent protein (XFP) marker. (We refer to this as a “Pair of Co-dominantly marked Knock-ins” or PCK, as the approach could in principle use any two co-dominant markers, not just XFPs.) For example, one knock-in mutant allele may contain a Red Fluorescent Protein (RFP) marker and the other knock-in mutant allele an Enhanced Yellow Fluorescent Protein (EYFP) marker. One then creates a population of animals that contain both the RFP and the EYFP alleles as well as an unmarked wild type allele (one could imagine approaches for labeling the wild-type allele a third color, but this is not essential). As fluorescent markers are co-dominant, using this pair of mutations one can unambiguously identify a subset of animals—the animals labeled by both RFP and EYFP—that do not contain a wild-type copy of the gene of interest and hence are sterile. One can also unambiguously identify any homozygous wild type individuals as they will be non-fluorescent. In addition, one will also observe animals that express only RFP or EYFP: these animals will contain a mixture of sterile RFP/RFP and EFYP/EFYP homozygotes as well as fertile heterozygotes. The sterile homozygotes will not reproduce, but the heterozygotes will permit maintenance of the strain and production of the next generation of sterile mutants. An advantage of the use of fluorescent labels is that automated fluorescence-based sorting strategies and equipment are currently available that are amenable to large-scale efforts. Furthermore, sorting is minimally invasive and does not cause significant reductions in viability, so one can imagine multiple rounds of sorting to increase purity of various populations. It is important to note that PCK differs from traditional use of a fluorescent knock-in, where both heterozygous and homozygous mutants are the same color, complicating unambiguous discrimination between them.

The present method relies on knocking out genes required for fertility on the autosomes making the method more broadly applicable in at least two ways. First, manipulating genes required for fertility can be readily applied to new isolates and species quickly, with limited understanding of the mechanisms of chromosomal segregation in the new organism. Second, the present method can be readily applied to insects which do not have distinct X and Y chromosomes, such as the major disease vector mosquito Aedes aegypti.

In general, a genetic strategy for sterile insect production commonly includes three components: 1) a strategy for creating males that are sterile but are otherwise sufficiently fit for mating, 2) a strategy for maintaining large numbers of animals that can generate these sterile males and 3) a strategy for unambiguously identifying the sterile individuals from within a population (for release), as well as identifying potentially fertile individuals heterozygous mutant for the gene required for fertility (to propagate the stock and produce future generations of sterile individuals). The PCK approach solves all three of these challenges. Importantly, while initially developed in Anopheles gambiae, PCK is readily generalizable to other species of insects for which there is genome sequence information and which can be genetically modified in a targeted fashion as well as grown in captivity. To apply the technique, one knocks-in XFPs under the control of a strong promoter into an endogenous gene required for fertility. In terms of the sterility genes to be targeted, these genes will show some variation among species, but such genes have been identified in flies and mosquitoes and are numerous and often exhibit significant evolutionary conservation, making it possible to identify multiple promising targets in a given arthropod species based on DNA sequence conservation.

In an aspect, a method of making sterile diploid organisms comprises
mating a first population of single knock-in diploid organisms and a second population of single knock-in diploid organisms,
   wherein the first population of single knock-in diploid organisms are heterozygous organisms expressing a first marker inserted into a gene required for fertility, wherein introduction of the first marker disrupts the function of the gene required for fertility creating a first mutant allele of the gene required for fertility,
   wherein the second population of single knock-in diploid organisms are heterozygous organisms expressing a second marker inserted into the gene required for fertility, wherein introduction of the second marker disrupts expression of the gene required for fertility creating a second mutant allele of the gene required for fertility, and
   wherein the first and second markers are distinct;
sorting offspring produced from the mating based on their expression of the first and/or second markers; and
isolating the sterile diploid organisms, wherein the sterile diploid organisms are heteroallelic diploid organisms expressing the first marker in the first mutant allele and the second marker in the mutant second allele of the gene required for fertility.

The methods described herein can be applied to any diploid organism. Specific diploid organisms include arthropods such as insects and arachnids.

Exemplary arthropods are of the genus Drosophila, Stegomyia, Aedes, Anopheles, Lutzomyia, Brumptomia, Warileya, Phlebotomus, Sergentiomyia, Cochliomyia, Chrysomyia, Glossinia, Ceratitis, Homalodisca, and Culex.

Specific examples of arthropods include Adelges piceae, Aedes aegypti, Aedes albopictus, Agrilus planipennis, Amblyomma americanum, Amblyomma maculatum, Anastrepha fraterculus, Anastrepha ludens, Anastrepha obliqua, Anastrepha suspense, Anopheles albimanus, Anopheles coluzzii, Anopheles freeborni, Anopheles gambiae, Anopheles quadrimaculatus, Anopheles stephensi, Anoplophora glabripennis, Bactrocera correcta, Bactrocera cucurbitae, Bactrocera dorsalis, Bactrocera oleae, Bactrocera philippinensis, Bactrocera tryoui, Bemisia tabaci, Cactoblastis cactorum, Ceratitis capitate, Ceutorhynchus obstricuts, Chrysodeixis includes, Chrysomya bezziana, Cimex hemipterus, Cimex lectularius, Cochliomyia hominivorax, Coptotermes formosanus, Culex pipiens, Culex quinquefasciatus, Culex tarsalis, Culiseta melanura, Cydia pomonella, Delia antiqua, Dermacentor variabilis, Diaphorina citri, Diuraphis noxia, Drosophila melanogaster, Drosophila suzukii, Drosophila pulchrella, Epiphyas postvittana, Haemagogus spp., Haematobia irritans, Halyomorpha halys, Homalodisca vitripennis, Hyalomma excavatum, Ixodes cookie, Ixodes pacificus, Ixodes scapularis, Lepeophtheirus salmonis, Leptotrombidium spp., Liponyssoides sanguineus, Lutzomyia longipalpis, Lycorma delicatula, Lymantria dispar, Megacopta cribraria, Ochlerotatus triseriatus (Aedes triseriatus), Orgyia anartoides, Panstrongylus megistus, Pectinophora gossypiella, Pediculus humanus, Phlebotomus argentipes, Phlebotomus papatasi, Plutella xylostella, Popillia japonica, Rhipicephalus sanguineus, Rhodnius prolixus, Scirtothrips dorsalis, Simulium spp, Solenopsis invicta, Somoxys calcitrans, Spodoptera frugiperda, Stegomyia aegypti, Tetropium fuscum, Thaumatotibia leucotreta, Triatoma brasiliensis, Triatoma dimidiate, Triatoma infestans, Trogoderma granarium, Tunga penetrans, and Xenopsylla cheopis. This extensive list of arthropods demonstrates that one of ordinary skill in the art could readily identify a target diploid organism of interest for use in the methods described herein.

Additional diploid organisms for use in the methods described herein include virtually any diploid organism amenable to transgenesis. Of particular interest is the generation of sterile organisms, including vertebrates, as biological control agents that can be released as a controlled and self-limited means of controlling the population of other species. Exemplary organisms include fish and rodent pests and rabbits, for example. For example, sterile carp can be released into bodies of water to mate with wild populations of carp as a means of population control. Sterile grass carp can be released as a method of vegetation control. Sterile tilapia eat algae. Sterile domesticated fish can be used to prevent contamination of native populations from domesticated fish that are released into the wild.

As used herein, the term “inserted” refers to the introduction of a heterologous recombinant nucleic acid sequence into the genome of the target organism.

In an aspect, first and second marker are inserted into the gene required for fertility at the same position. However, it is also possible that the first and second marker are inserted into the gene required for fertility to limit or prevent genetic recombination between the inserted markers, such as insertion of each marker within the exons or regulatory regions of the gene. Yet further, the first and second marker may be inserted into the gene required for fertility such that the knock-ins create one or more copies of the required fertility gene containing a deletion or rearrangement.

Advantageously, the heterozygotes will permit maintenance of the strain and production of the next generation of sterile mutants. Thus, in an aspect, the method further comprises isolating offspring expressing the first marker only to provide a fertile first population of single knock-in organisms, isolating offspring expressing the second marker only to provide a fertile second population of single knock-in organisms, or both.

In an aspect, the first population of single knock-in organisms, the second population of single knock-in organisms, or both, also contains a third marker expressed on the X chromosome, or in a sex-determination gene.

Exemplary markers for the first, second and third markers include fluorescent protein markers (e.g., red fluorescent protein, enhanced yellow fluorescent protein, green fluorescent protein, and the like), a drug resistance marker (e.g., puromycin N-acetyltransferase which provides resistance to puromycin; the tetA gene which provides resistance to tetracycline; the Sh ble gene which provides resistance to zeocin; and the like), aminoglycoside phosphotransferases (which provide resistance to geneticin (G418), neomycin, kanamycin, and the like), or a combination thereof.

Fertility genes in arthropods include genes that have a sterile phenotype when mutated in the arthropod.

Exemplary genes that have been demonstrated to be required for fertility in arthropods include (using the names of the Drosophila melanogaster orthologs) Zero Population growth, PFTAIRE interacting factor 1A, Sperm-Leucylaminopeptidase 8, Merry-go-round, myo-inositol-1-phosphate synthase, no mitochondrial derivative, male sterile (3), ADP ribosylation factor at 51F, GLD2 poly(A) polymerase, RNA 3′-terminal phosphate cyclase, Sperm-Leucylaminopeptidase 2, valois, male sterile (2) 34Fe, Brunelleschi, CG31759, Proteasome α6 subunit, Testis-specific, CDP-diacylglycerol synthase, doublefault, no child left behind, mitoferrin, RNaseP protein p30, TBP-associated factor 6, centrosomin, Gamma-tubulin ring protein 84, F-box synaptic protein, transformer-2, Myb-interacting protein 120, blanks, bag of marbles, male sterile (3) K81, Dynein intermediate chain at 61B, misfire, minotaur, kelch like family member 10, Sperm-Leucylaminopeptidase 3, spindle E, Dis3 like 3′-5′ exoribonuclease 2, eukaryotic translation release factor 3, male sterile (2) 35Ci, Zeste-white 10, australin, wuho, Radial spoke head protein 1, subito, spindle defective 2, Apollo, stem cell tumor, Sauron, Regulator of cullins 1b, Radial spoke head protein 3, dilute class unconventional myosin, Ubiquitin specific protease 14, Transcription factor B5, Vacuolar H+ ATPase PPA1 subunit 2, small nuclear RNA U7, Sak kinase, scotti, BRCA2 DNA repair associated, Dynein light chain 90F, maelstrom, karyopherin al, Polycystic kidney disease 2, Dead-box-1, shutdown, eukaryotic translation initiation factor 4G2, Sperm-Leucylaminopeptidase 5, gudu, paired, goddard, meiosis I arrest, thoc5, aegypt onions, protamine A, Male-specific transcript 77F, Grip75, papi, traffic jam, Spindle assembly abnormal 4, mulet, cinnabar, SR Protein Kinase, Radial spoke binding protein 15, Syntaxin 5, male fertility factor kl5, infertile crescent, male fertility factor kl3, sneaky, deadlock, nutcracker, twine, Phosphatidic Acid Phospholipase A1, garnet, Trapped in endoderm 1, testis-specifically expressed bromodomain containing protein-1, Cytokine induced apoptosis inhibitor 1, lost, Radial spoke head protein 9, big bubble 8, long non-coding RNA:iab8, HBS1, SHC-adaptor protein, Sperm-Leucylaminopeptidase 7, nebbish, four wheel drive, meiotic from via Salaria 332, no hitter, boule, Angiotensin converting enzyme, aubergine, novel, spermatogenesis regulator, eukaryotic translation initiation factor 3 subunit m, P-element induced wimpy testis, male sterile (3) 76Ca, don juan, Chromodomain-helicase-DNA-binding protein 1, CG13202, effete, La related protein, tombola, puffyeye, Basal body up regulated gene 22, oskar, Growth arrest specific protein 8, belle, milkah, Peroxin 12, Centrosomal protein 135 kDa, Cytochrome c distal, distal antenna-young, Reduction in Cnn dots 7, Topoisomerase I-interacting protein, Hephaestus, Myb-interacting protein 40, Kinesin-like protein at 3A, salto, james bond, CG18675, lodestar, deformed wings, combover, loopin-1, Sperm-Leucylaminopeptidase 1, male sterile (3) 76Cc, asunder, defective transmitter release, JYalpha, male fertility factor kl2, always early, pelota, mitochondrial Cytochrome b, Cyclin B, claret, tudor, Kinesin-like protein at 67A, Cytochrome b5, NADH dehydrogenase (ubiquinone) B17 subunit, exuperantia, TBP-related factor, testis-specifically expressed bromodomain containing protein-2, multi sex combs, Peroxin 10, Gamma-tubulin ring protein 91, Symplekin, Oxen, xmas, Grip128, Deadbeat, U2A, Neurotransmitter transporter-like, Capsuleen, Sperm-Leucylaminopeptidase 4, Ubiquitin-63E, β-Tubulin at 85D, suppressor of sable, Dpy-30-like 2, knotted onions, noisette, Dynactin 5, p25 subunit, Deubiquitinating enzyme A, ovarian tumor, benign gonial cell neoplasm, meiotic P26, Tubulin tyrosine ligase-like 3B, Cannonball, mitoshell, Peptidyl-α-hydroxyglycine-α-amidating lyase 1, Syntaxin 13, Vreteno, eukaryotic translation initiation factor 4E3, sheepish, scattered, tumorous testis, matotopetli, Radial spoke head protein 4a, spermatocyte arrest, sperm-associated antigen, an ortholog or a homolog thereof. This extensive list of genes demonstrated to be required for fertility in Drosophila melanogaster demonstrate that one of ordinary skill in the art could readily identify a gene required for fertility in any diploid organism of interest.

The introduction of markers or “gene editing” provides the ability to manipulate the DNA sequence of a cell at a specific chromosomal locus. This technology effectively enables manipulation of the genome of a subject's cells in vitro or in vivo.

In an aspect, gene editing involves targeting an endonuclease (an enzyme that causes DNA breaks internally within a DNA molecule) to a specific site of the genome and thereby triggering formation of a chromosomal double strand break (DSB) at the chosen site. An endonuclease is an enzyme that cleaves the phosphodiester bond within a polynucleotide chain, such as DNA. If, concomitant with the introduction of the chromosome breaks, a donor DNA molecule is introduced (for example, by plasmid or oligonucleotide introduction), interactions between the broken chromosome (DNA) and the introduced DNA can occur, especially if the two sequences share homology. In this instance, a process termed “gene targeting” can occur, in which the DNA ends of the chromosome invade homologous sequences of the donor DNA by homologous recombination (HR). By using the donor plasmid sequence as a template for HR, a seamless repair of the chromosomal DSB can be accomplished. Importantly, if the donor DNA molecule differs slightly in sequence from the chromosomal sequence, HR-mediated DSB repair will introduce the donor sequence into the chromosome, resulting in gene conversion/gene correction of the chromosomal locus. By targeting the nuclease to a genomic site of interest, the concept is to use DSB formation to stimulate HR and to thereby replace target sequence with a desired sequence, which might include a gene having a deletion or mutation (e.g., insertion, point mutation, frame shift, or a larger insertion or deletion).

In an aspect, in the methods described herein, the first population of single knock-in organisms is produced by injecting insect embryos with plasmid(s) that express a protein and a nucleic acid (e.g., Cas9 in combination with an appropriate guideRNA) or pre-made protein/nucleic acid (e.g., Cas9/guideRNA) complexes that together produce a targeted DNA break at the site of interest (in a gene required for fertility, at a location (or locations) where insertion or deletion of DNA will disrupt its function) as well as a first homologous repair vector, wherein the first homologous repair vector directs gene insertion and expression of the first marker in the first allele of the gene required for fertility; and the second population of single knock-in organisms is produced by injecting insect embryos with plasmid(s) that express a protein and a nucleic acid (e.g., Cas9 in combination with an appropriate guideRNA) or pre-made protein/nucleic acid (e.g., Cas9/guideRNA) complexes that together produce a targeted DNA break at the site of interest (in a gene required for fertility, at a location (or locations) where insertion or deletion of DNA will disrupt its function) as well as with a second homologous repair vector, wherein the second homologous repair vector directs gene insertion and expression of the second marker in the second allele of the gene required for fertility.

Genome editing tools use the induction of double strand breaks (DSBs) to enhance gene manipulation of cells. Such methods include zinc finger nucleases (ZFNs), Transcription Activator-Like Effector Nucleases (TALENs), and the CRISPR (Clustered Regularly Interspaced Short Palindromic Repeats)/Cas9 system.

RNA-guided nuclease-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break (DSB). The DSB is then repaired either by non-homologous end joining (NHEJ), which leads to insertion/deletion (indel) mutations, or by homology-directed repair (HDR), which requires an exogenous template and can generate a precise modification at a target locus. Unlike other gene editing methods, which add a functional, or partially functional, copy of a gene to a subject's cells but retain the original copy of the gene, this system can remove and replace the target gene. Genetic editing using engineered nucleases has been demonstrated in tissue culture cells and in vivo.

Cas9 is a nuclease, an enzyme specialized for cutting DNA, with two active cutting sites, one for each strand of the double helix. It has been demonstrated that one or both sites could be disabled while preserving Cas9's ability to home locate its target DNA. TracrRNA and spacer RNA can be combined into a “single-guide RNA” molecule that, when mixed with Cas9, can find and cut the correct DNA targets.

As an RNA guided protein, Cas9 requires a short RNA to direct the recognition of DNA targets. Though Cas9 preferentially interrogates DNA sequences containing a PAM sequence, NGG, it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs, the synthetic construct gRNA was created to combine the essential pieces of RNA for Cas9 targeting into a single RNA expressed with the RNA polymerase type 21 promoter (U6). Synthetic gRNAs are slightly over 100 bp at the minimum length and contain a portion which targets the 20 protospacer nucleotides immediately preceding the PAM sequence NGG; gRNAs do not contain a PAM sequence.

In an aspect, the genome targeting vectors each encode Cas9 and a first or a second gRNA, respectively, wherein the gRNAs bind the Cas9 and comprise a target sequence in the gene required for fertility; wherein the genomic insertions of the first and second markers are done using an endonuclease that makes double stranded chromosomal breaks; wherein the genomic insertions of the first and second markers are done using zinc finger nucleases. In another aspect, the genomic insertions of the first and second markers are done using Transcription Activator-Like Effector Nucleases; or a combination thereof.

When the first and second markers comprise fluorescent proteins, sorting offspring produced from the mating based on their expression of the first and/or second markers can be done using FACS sorting. Fluorescence activated cell sorting (FACS) of live animals separates a population into sub-populations based on fluorescent labeling.

Alternatively, the first and second markers can comprise antibiotic resistance markers.

Also included herein are sterile insects produced by any of the methods disclosed herein.

In an aspect, a method of mitigating or eradicating arthropods in an arthropod population comprises releasing into the arthropod population sterile arthropods produced by the methods described herein. In an aspect, the arthropod population is a wild arthropod population.

In another aspect, a method of eradicating or mitigating diploid organisms in a diploid organism population comprises releasing into the diploid organism population a population of heteroallelic sterile diploid organisms expressing first and second markers, wherein introduction of the first marker disrupts function of a gene required for fertility to create a first mutant allele of the gene required for fertility, and introduction of the second marker disrupts function of the gene required for fertility to create a second mutant allele of the gene required for fertility, and wherein the first and second markers are different. In an aspect, the diploid organisms are arthropods. In an aspect, the arthropod population is a wild arthropod population.

In an aspect, wherein the arthropod population comprises a crop or wildlife pest, and the sterile arthropods are sex sorted. In another aspect, the arthropod population comprises a crop or wildlife pest, and the sterile arthropods are not sex sorted. Crop and wildlife pests include the arthropods described herein.

In an aspect, the heteroallelic sterile diploid organisms are sex-sorted males, and wherein the sterile males shorten the lifespan of females to which they mate, suppress egg production of the females to which they mate, or a combination thereof.

In an aspect, the heteroallelic sterile diploid organisms are sex-sorted males, and wherein the sterile males increase the rate of unhatched eggs by mating with wild-type female insects.

In an aspect, the arthropod population comprises an arthropod disease vector, and the sterile arthropods are sex sorted. In another aspect, wherein the arthropod population comprises an arthropod disease vector, and the sterile arthropods are not sex sorted. Arthropod disease vectors are described herein. Exemplary diseases that are mosquito-borne include malaria, Dengue, chikungunya and Zika virus infections.

Of particular relevance to the sterile insect technique, sterile males are expected to mate with wild females, leading to a reduction in the number of progeny produced by the population of wild females. Releases can include both males and females, although some programs utilize just one sex, and this is typically due to specific biological or ecological requirements.

Depending on the lifespan and mating frequency of the target arthropod, a pulsed release schedule may be employed to provide optimal reduction/elimination of the wild insect population.

Compared to the prior art irradiation and chemical sterilization, the PCK-based genetic sterilization can be tissue-specific in its effects, allowing one to achieve extreme sterilization with fewer fitness effects than whole-animal treatments with radiation or chemicals.

For example, compared to prior art Wolbachia infection, the PCK-based genetic sterilization can be achieved by targeting evolutionarily conserved genes, making it more readily applicable to more species than Wolbachia, which does not readily infect all insects. Genetic sterilization is also unlikely to promote resistance in the target population of insects. This contrasts with Wolbachia, which can lose its effectiveness if the introduced Wolbachia-containing populations take hold in the wild. Finally, genetic sterilization should act in a species-specific fashion. In contrast, releasing large quantities of Wolbachia into the environment could potentially impact other susceptible insect species and not just the target species.

Further, compared to the prior art method of Release of Insects carrying a Dominant Lethal (RIDL), PCK-based genetic sterilization is readily applicable to many species without the need for introducing, developing and titrating poison constructs. In addition, PCK-based genetic sterilization is carried out in the genetic background of the population grown for creating the sterile individuals, while RIDL occurs in a mixed genetic background (half the background comes from the RIDL strain and half from the polymorphic wild population with which the RIDL individuals mate). The mixed genetic background could impair RIDL's effectiveness.

Yet further, compared to the prior art pgSIT method which has been proposed but not field-tested, PCK-based genetic sterilization creates a stable genetically defined population of fully mutant and hence sterile individuals, while pgSIT generates a heterogenous mix of individuals without defined genetic lesions. Additionally, in the PCK approach sterile animals are unambiguously identified from a single mating population, while the pgSIT strategy requires accurate sexing to set up new crosses at each generation. This is more labor intensive and provides more opportunity for errors as any defects in sexing of parents will lead to the environmental release of non-sterile, Cas9-expressing transgenic individuals.

Finally, compared to the gene-drive method which proposed but not yet field-tested, PCK-based genetic sterilization is self-limiting and does not result in the transmission of transgenic DNA (or organisms in the case of Wolbachia) within wild populations. This makes it a local and environmentally friendly strategy. Gene-drives, on the other hand, use genetic approaches to bias inheritance of alleles in an attempt to replace, or even eliminate target species. In the extreme case, the drive could spread through the population, causing the extinction of a species. Alternatively, gene drives could result in introduction of transgenic material into wild populations either by design, accidental release of non-sterile offspring, or due to evolved resistance and breakdown of the genetic drive. Each of these cases could lead to unforeseen environmental impacts that are avoided by the PCK approach.

In another aspect, the population of heteroallelic sterile diploid organisms are biological control agents of a species different from the population targeted for population control. In this aspect, a self-limiting population of heteroallelic sterile diploid biological control agents can be released to reduce the target organism population. Thus, in an aspect, the heteroallelic sterile diploid organisms are biological control agents for the diploid organism population that is a different species than the heteroallelic sterile diploid organisms. Because the heteroallelic sterile diploid biological control agents cannot reproduce, there is no danger of the biological control agents reproducing or becoming an established population.

In an example, predator sterile mosquitofish (Gambusia affinis), fat head minnow and dragonflies which can eat mosquito larvae, controlling mosquito populations.

The invention is further illustrated by the following non-limiting examples.

### EXAMPLES

### Materials and Methods

Animal Rearing and Stock Maintenance: Anopheles gambiae G3 strain was used as wild-type and genetic background for transgenesis. Animals were raised in mesh cages (Bugdorm, MegaView Science Co., Ltd., Taiwan) in incubators (Percival Scientific) set to 25-28° C. and 70-80% humidity with a 12 h light:12 h dark cycle. Adults were supplied with a water source and 10% w/v glucose ad libitum. Blood feeding was performed with human blood (Research Blood Components, Watertown, MA) warmed using a membrane feeding system (Hemotek, ltd., Blackburn, UK). Feeding procedures were approved and monitored by Brandeis Institutional Biosafety Committee. Larva were kept at a density of approximately 200 animals/tray in deionized water and fed a mixture of powdered fish food (TetraMin flakes #77101 and TetraPond sticks #16467, Tetra Co., Melle, Germany) and Koi pellets (Koi's Choice #100033588, Kaytee Products, Inc., Chilton, WI).

Vector Design: To generate a vector encoding both Cas9 and gRNA, site directed mutagenesis to eliminate the Bsa1 cut site from pUC19 was performed according to manufacturer's instructions (QuikChange® Lightning; Agilent Technologies) using primers

SEQ ID NO: 1
5′-GCAATGATACCGCGGGATCCACGCTCACCGGCTCC-3′
and
SEQ ID NO: 2
5′-GGAGCCGGTGAGCGTGGATCCCGCGGTATCATTGC-3′.

Anopheles U6 promoter and sgRNA scaffold were PCR amplified from P125-pBac[3xp3-RFP]Attp-U6-gRNA-Eco31 (a gift from Andrew Hammond, Crisanti Laboratory, Imperial College London) using primers 5′-GCCAGGACGTCCTTTGTATGCGTGCGCTTGAAG-3′ SEQ ID NO: 3 and 5′-CCGTATAAGTTCGAGATCGGCC-3′ SEQ ID NO: 4. A fragment encoding human-codon-optimized Cas9 under the control of the germline specific vasa2 promoter and 3′UTR was cut from 155-attB-CFP-Vas2-hCas9-Vas3utr (a gift from Andrew Hammond, Crisanti Laboratory) with Asc1 and SbfI and all fragments combined using standard molecular cloning methods.

gRNAs devoid of predicted off-target sites in the Anopheles genome were identified using CRISPR Optimal Target Finder. Complimentary oligonucleotides corresponding to the target site were synthesized with complimentary overhangs (Eton Bioscience, Charlestown, MA), annealed, and cloned into the Vasa-Cas9-sgRNA backbone vector using Bsa1 restriction sites.

Generation of IR93a^{EYFP/RFP} mosquitoes: Blood-fed and mated G3 females were placed in embryo collection tubes consisting of an inverted 50 mL conical tube with the tip removed and covered with tulle mesh for air ventilation and a water-saturated piece of filter paper placed in the cap. These were stored in a 28° C. incubator for about 20 minutes after which time the filter paper was removed and embryos were transferred to a glass slide. Embryos were positioned on the edge of a nitrocellulose membrane (45 um pore size, Life Technologies, Carlsbad, CA) that was topped by a wet piece of extra thick western blotting filter paper so that a thin meniscus of water formed around them.

Injection needles were pulled on a Model P-97 Flaming/Brown Micropipette Puller from Sutter Instrument Co using aluminosilicate capillaries (Sutter Instruments Item #AF100-64-10). Program: Heat=547, Pull=50, Vel.=60, Del.=90, Ramp=557, P=300. Needles were beveled (BV10, Sutter Instruments) a 20° angle using bubbles from pressurized air to monitor opening size. IR93a targeting vector (300 ng/ul) and donor vectors (RFP and EYFP, 150 ng/ul each) were injected simultaneously into 1365 embryos. Injections took place on an inverted microscope using a PLI-100 picoinjector (Harvard Apparatus) with the balance pressure calibrated to produce a constant outflow of injection mix.

Surviving injected adults were mated with G3 and the progeny of this cross were screened for fluorescence. Fluorescent F1 animals were outcrossed to G3 for three more generations before creating a homozygous stock. To genotype, animals were briefly anesthetized on ice and a single leg was removed and placed in an individual PCR tube. Individual animals were kept in 3 fl oz Dixie cups with a tulle covering, provided with sugar and water saturated cotton balls, and stored in incubators while awaiting the results of PCR. Each leg was incubated at 37° C. overnight in DNA extraction buffer (10 mM Tris HCl, pH 8.2, 1 mM EDTA, 25 mM NaCl with proteinase K (200 mg/mL). PCR with Taq polymerase (NEB) was performed using one universal forward primer and two reverse primers corresponding to the wild type or insert sequence (Universal forward primer 5′-CACATCACATCACAAGGAGTGC-3′ SEQ ID NO: 5, WT 5′-TAGGAAAGGTTAGAAAAGCGAC-3′ SEQ ID NO: 6, Mutant 5′-CCGTATTGGCCACGTGTCC-3′ SEQ ID NO: 7). PCR products were run on a 1% agarose gel, and WT and mutant alleles differentiated based on size differences (605 bp for WT and 478 bp for mutant).

### Example 1: Two Color Knock-In of Ir93a

IR93a is an ionotropic receptor gene from An. gambiae. Knock-ins to the IR93a gene were produced in RFP and EYFP versions. As shown in FIG. 1, by creating a pair of knock-in alleles identical except for their fluorescent label, one can unambiguously identify animal carrying two mutant alleles: RFP/YFP co-expression indicates both the RFP-linked knock-in allele and the EYFP-linked knock-in allele are present and also indicates that the animal does not include a wild type allele. These double-knock-in animals lack IR93a gene function.

### Example 2: Homozygous Mutant Animals can be Identified at Multiple Life Stages

A key aspect of the PCK strategy is that it allows one to unambiguously identify sterile animals (that carry two copies of a recessive mutation in a target gene important for fertility) from a heterogeneous population of animals, many of whom are fertile because they contain one or two wild type copies of the target gene. In this way, PCK allows one to select sterile animals (for potential release) while continuing to propagate the mutation-containing population, enabling future rounds of sterile insect production and release. An important practical consideration is that for different applications it may be optimal to isolate mutant animals at different life stages prior to release. For example, for some applications (or species) it may be optimal to isolate mutants from the general population at early larval stages, while for others it may be optimal to perform such isolation at the adult stage.

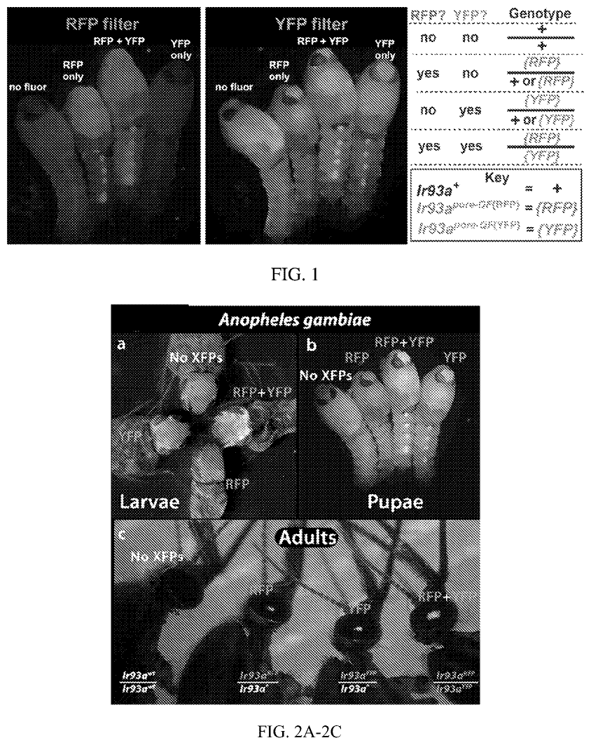

FIG. 2 demonstrates that PCK can be used to identify homozygous mutant animals at multiple different life stages. Specifically, FIG. 2 demonstrates that PCK can be used to uniquely identify homozygous mutant animals in the malaria vector Anopheles gambiae based on co-expression of RFP and EYFP at three different life stages, larval, pupal and adult. The specific life stages at which PCK can be used depends on the specific transcriptional promoter chosen to drive marker expression. In FIG. 2, the 3XP3 promoter is used to drive XFP (e.g., RFP and EYFP) expression. 3XP3 drives expression in the visual system across a wide range of insects and developmental stages, from larval to pupal to adult, as illustrated for Anopheles gambiae in FIG. 2. Other transcriptional promoters that are expressed in different anatomical patterns, at different life stages and/or different species can be readily substituted for 3XP3 using established molecular biology techniques. Thus, PCK is not limited in the life stages at which it can be used to identify homozygous mutant animals.

### Example 3: The PCK Strategy can be Used in Multiple Species

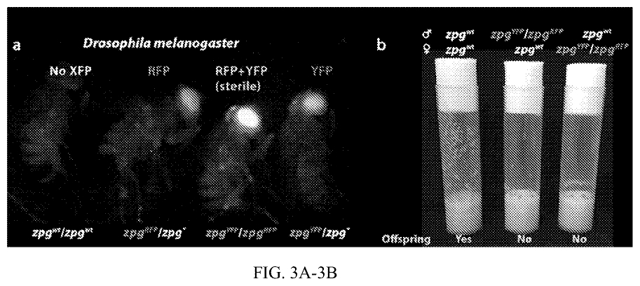

The initial implementation of PCK was in the malaria mosquito Anopheles gambiae. A strength of PCK is that it can be readily adapted for use in other species. To demonstrate this, PCK was used in Drosophila melanogaster, creating XFP (e.g., RFP or EYFP) disruptions of zpg (zero population growth), a gene required for fertility. As shown in FIG. 3a, zpg^{RFP}/zpg^{YFP} animals (animals containing one zpg^{RFP} and one zpg^{YFP} disruption allele) can be uniquely distinguished from potential zpg heterozygotes and wild type animals based on their co-expression of both RFP and EYFP. As predicted given the requirement for zpg activity in both male and female, both male and female zpg^{RFP}/zpg^{YFP} mutant flies were sterile (FIG. 3b). As illustrated in FIG. 3b, when wild type males were placed in the same vial of food as virgin flies, allowing them to mate, numerous progeny were generated. In contrast, zpg^{RFP}/zpg^{YFP} males placed with wild type virgin females yielded no progeny. Similarly, zpg^{RFP}/zpg^{YFP} females placed with wild type males also yielded no progeny. This demonstrates the ability to generate sterile males and sterile females using the PCK approach.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, alleles. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of making sterile diploid organisms comprising
mating a first population of single knock-in diploid organisms and a second population of single knock-in diploid organisms,
wherein the first population of single knock-in diploid organisms are heterozygous organisms expressing a first marker inserted into a gene required for fertility,
wherein introduction of the first marker disrupts the function of the gene required for fertility creating a first mutant allele of the gene required for fertility,
wherein the second population of single knock-in diploid organisms are heterozygous organisms expressing a second marker inserted into the gene required for fertility, wherein introduction of the second marker disrupts expression of the gene required for fertility creating a second mutant allele of the gene required for fertility, and
wherein the first and second markers are distinct;
sorting offspring produced from the mating based on their expression of the first and/or second markers; and
isolating the sterile diploid organisms, wherein the sterile diploid organisms are heteroallelic diploid organisms expressing the first marker in the first mutant allele and the second marker in the mutant second allele of the gene required for fertility.

2. The method of claim 1, wherein the diploid organism is an arthropod, specifically an insect or an arachnid.

3. The method of claim 1, wherein the first and second marker are inserted into the gene required for fertility at the same position; wherein the first and second marker are inserted into the gene required for fertility to limit or prevent genetic recombination between the inserted markers, such as each marker is inserted within the exons or regulatory regions of the gene; or wherein the first and second marker are inserted into the gene required for fertility such that the knock-ins create one or more copies of the gene required for fertility containing a deletion or rearrangement.

4. The method of claim 1, further comprising isolating offspring expressing the first marker only to provide a fertile first population of single knock-in organisms, isolating offspring expressing the second marker only to provide a fertile second population of single knock-in organisms, or both.

5. The method of claim 2, wherein the insect comprises an insect of the genus Drosophila, Stegornyia, Aedes, Anopheles, Lutzomyia, Brumptomia, Warileya, Phlebotomus, Sergentiomyia, Cochliomyia, Chrysomyia, Glossinia, Ceratitis, Homalodisca, or Culex.

6. The method of claim 2, wherein the insect comprises Adelges piceae, Aedes aegypti, Aedes albopictus, Agrilus planipennis, Amblyomma americanum, Amblyomma maculatum, Anastrephafraterculus, Anastrepha ludens, Anastrepha obliqua, Anastrepha suspense, Anopheles albimanus, Anopheles coluzzii, Anopheles freebomi, Anopheles gambiae, Anopheles quadrimaculatus, Anopheles stephensi, Anoplophora glabripennis, Bactrocera correcta, Bactrocera cucurbitae, Bactrocera dorsalis, Bactrocera oleae, Bactrocera philippinensis, Bactrocera tryoni, Bemisia tabaci, Cactoblastis cactorum, Ceratitis capitate, Ceutorhynchus obstricuts, Chrysodeixis includes, Chrysomya bezziana, Cimex hemipterus, Cimex lectularius, Cochliomyia hominivorax, Coptotermes formosanus, Culex pipiens, Culex quinquefasciatus, Culex tarsalis, Culiseta melanura, Cydia pomonella, Delia antiqua, Dermacentor variabilis, Diaphorina citri, Diuraphis noxia, Drosophila melanogaster, Drosophila suzukii, Drosophila pulchrella, Epiphyas postvittana, Haemagogus spp., Haematobia irritans, Halyomorpha halys, Homalodisca vitripennis, Hyalomma excavatum, Ixodes cookie, Ixodes pacificus, Ixodes scapularis, Lepeophtheirus salmonis, Leptotrombidium spp., Liponyssoides sanguineus, Lutzomyia longipalpis, Lycorma delicatula, Lymantria dispar, Megacopta cribraria, Ochlerotatus triseriatus (Aedes triseriatus′), Orgyia anartoides, Panstrongylus megistus, Pectinophora gossypiella, Pediculus humanus, Phlebotomus argentipes, Phlebotomus papatasi, Plutella xylostella, Popillia japonica, Rhipicephalus sanguineus, Rhodnius prolixus, Scirtothrips dorsalis, Simulium spp, Solenopsis invicta, Somoxys calcitrans, Spodoptera frugiperda, Stegornyia aegypti, Tetropium fuscum, Thaumatotibia leucotreta, Triatoma brasiliensis, Triatoma dimidiate, Triatoma infestans, Trogoderma granarium, Tunga penetrans, or Xenopsylla cheopis.

7. The method of claim 1, wherein the first population of single knock-in organisms, the second population of single knock-in organisms, or both, further comprises a third marker expressed on the X chromosome, or in a sex-determination gene.

8. The method of claim 1,
wherein the first population of single knock-in organisms is produced by injecting embryos with a first homologous repair vector, wherein the first homologous repair vector directs gene insertion and expression of the first marker in the first allele of the gene required for fertility; and
wherein the second population of single knock-in organisms is produced by injecting embryos with a second homologous repair vector, wherein the second homologous repair vector directs gene insertion and expression of the second marker in the second allele of the gene required for fertility.

9. The method of claim 1, wherein the first and second homologous repair vectors are used in combination with a genome targeting vector that encodes Cas9 and a first or a second gRNA, respectively, or in combination with pre-formed Cas9/gRNA complexes, wherein the gRNAs and Cas9 complex to target a sequence in the gene required for fertility; wherein the gene insertion from the first and second homologous repair vectors is done using an endonuclease that makes double stranded chromosomal breaks; wherein the gene insertion from the first and second homologous repair vectors is done using zinc finger nucleases; wherein the gene insertion from the first and second homologous repair vectors is done using Transcription Activator-Like Effector Nucleases; or a combination thereof.

10. The method of claim 2, wherein the gene required for fertility in the arthropod is a gene that has a sterile phenotype when mutated in the arthropod.

11. The method of claim 10, wherein the gene required for fertility in the arthropod is Zero Population growth, PFTAIRE interacting factor 1A, Sperm-Leucylaminopeptidase 8, Merry-go-round, myo-inositol-1-phosphate synthase, no mitochondrial derivative, male sterile (3), ADP ribosylation factor at 51F, GLD2 poly(A) polymerase, RNA 3′-terminal phosphate cyclase, Sperm-Leucylaminopeptidase 2, valois, male sterile (2) 34Fe, Brunelleschi, CG31759, Proteasome α6 subunit, Testis-specific, CDP-diacylglycerol synthase, doublefault, no child left behind, mitoferrin, RNaseP protein p30, TBP-associated factor 6, centrosomin, Gamma-tubulin ring protein 84, F-box synaptic protein, transformer-2, Myb-interacting protein 120, blanks, bag of marbles, male sterile (3) K81, Dynein intermediate chain at 6 IB, misfire, minotaur, kelch like family member 10, Sperm-Leucylaminopeptidase 3, spindle E, Dis3 like 3′-5′ exoribonuclease 2, eukaryotic translation release factor 3, male sterile (2) 35Ci, Zeste-white 10, australin, wuho, Radial spoke head protein 1, subito, spindle defective 2, Apollo, stem cell tumor, Sauron, Regulator of cullins 1b, Radial spoke head protein 3, dilute class unconventional myosin, Ubiquitin specific protease 14, Transcription factor B5, Vacuolar H+ ATPase PPA1 subunit 2, small nuclear RNA U7, Sak kinase, scotti, BRCA2 DNA repair associated, Dynein light chain 90F, maelstrom, karyopherin al, Polycystic kidney disease 2, Dead-box-1, shutdown, eukaryotic translation initiation factor 4G2, Sperm-Leucylaminopeptidase 5, gudu, paired, goddard, meiosis I arrest, thoc5, fuzzy onions, protamine A, Male-specific transcript 77F, Grip75, papi, traffic jam, Spindle assembly abnormal 4, mulct, cinnabar, SR Protein Kinase, Radial spoke binding protein 15, Syntaxin 5, male fertility factor kl5, infertile crescent, male fertility factor kl3, sneaky, deadlock, nutcracker, twine, Phosphatidic Acid Phospholipase A1, garnet, Trapped in endoderm 1, testis-specifically expressed bromodomain containing protein-1, Cytokine induced apoptosis inhibitor 1, lost, Radial spoke head protein 9, big bubble 8, long non-coding RNA:iab8, HBS1, SHC-adaptor protein, Sperm-Leucylaminopeptidase 7, nebbish, four wheel drive, meiotic from via Salaria 332, no hitter, boule, Angiotensin converting enzyme, aubergine, novel, spermatogenesis regulator, eukaryotic translation initiation factor 3 subunit m, P-element induced wimpy testis, male sterile (3) 76Ca, don juan, Chromodomain-helicase-DNA-binding protein 1, CG13202, effete, La related protein, tombola, puffyeye, Basal body up regulated gene 22, oskar, Growth arrest specific protein 8, belle, milkah, Peroxin 12, Centrosomal protein 135 kDa, Cytochrome c distal, distal antennayoung, Reduction in Cnn dots 7, Topoisomerase I-interacting protein, Hephaestus, Myb-interacting protein 40, Kinesin-like protein at 3A, salto, James bond, CG18675, lodestar, deformed wings, combover, loopin-1, Sperm-Leucylaminopeptidase 1, male sterile (3) 76Cc, asunder, defective transmitter release, JYalpha, male fertility factor kl2, always early, pelota, mitochondrial Cytochrome b, Cyclin B, claret, tudor, Kinesin-like protein at 67A, Cytochrome b5, NADH dehydrogenase (ubiquinone) B17 subunit, exuperantia, TBP-related factor, testis-specifically expressed bromodomain containing protein-2, multi sex combs, Peroxin 10, Gamma-tubulin ring protein 91, Symplekin, Oxen, xmas, Grip128, Deadbeat, U2A, Neurotransmitter transporter-like, Capsuleen, Sperm-Leucylaminopeptidase 4, Ubiquitin-63E, β-Tubulin at 85D, suppressor of sable, Dpy-30-like 2, knotted onions, noisette, Dynactin 5, p25 subunit, Deubiquitinating enzyme A, ovarian tumor, benign gonial cell neoplasm, meiotic P26, Tubulin tyrosine ligase-like 3B, Cannonball, mitoshell, Peptidyl-α-hydroxyglycine-α-amidating lyase 1, Syntaxin 13, Vreteno, eukaryotic translation initiation factor 4E3, sheepish, scattered, tumorous testis, matotopetli, Radial spoke head protein 4a, spermatocyte arrest, sperm-associated antigen, an ortholog or a homolog thereof.

12. The method of claim 1, wherein the first and second markers comprise a fluorescent protein marker, a drug resistance marker, an aminoglycoside phosphotransferase, or a combination thereof.

13. The method of claim 1, wherein the first and second marker comprise fluorescent proteins, and sorting comprises FACS sorting.

14. The method of claim 1, wherein the first and second marker comprise antibiotic resistance markers.

15. A sterile diploid organism population produced by the method of any of claim 1.

16. A progeny of arthropod eggs produced by the method of claim 4.

17. A method of mitigating or eradicating arthropods in an arthropod population, comprising releasing into the arthropod population sterile arthropods produced by the methods of claim 1.

18. A method of mitigating or eradicating diploid organisms in a diploid organism population, comprising releasing into the diploid organism population a population of heteroallelic sterile diploid organisms expressing first and second markers,
wherein introduction of the first marker disrupts function of a required fertility gene to create a first mutant allele of the gene required for fertility, and introduction of the second marker disrupts function of the required fertility gene to create a second mutant allele of the gene required for fertility, and
wherein the first and second markers are different.

19. The method of claim 18, wherein mating of the population of heteroallelic sterile diploid organisms and the diploid organism population reduces a number of viable progeny in a subsequent generation compared to release of a control insect.

20. The method of claim 18, wherein the heteroallelic sterile diploid organisms are sex-sorted males, and wherein the sterile males shorten the lifespan of females to which they mate, suppress egg production of the females to which they mate, or a combination thereof.

21. The method of claim 18, wherein the diploid organisms are arthropods.

22. The method of claim 21, wherein the arthropod population comprises a crop or wildlife pest, and the sterile arthropods are sex sorted.

23. The method of claim 21, wherein the arthropod population comprises a crop or wildlife pest, and the sterile arthropods are not sex sorted.

24. The method of claim 21, wherein the arthropod population comprises an arthropod disease vector, and the sterile arthropods are sex sorted.

25. The method of claim 21, wherein the arthropod population comprises an arthropod disease vector, and the sterile arthropods are not sex sorted.

26. The method of claim 18, wherein the heteroallelic sterile diploid organisms are biological control agents for the diploid organism population that is a different species than the heteroallelic sterile diploid organisms.

27. The method of claim 18, wherein the heteroallelic sterile diploid organisms are sex-sorted male insects, and wherein the sterile males increase the rate of unhatched eggs by mating with wild-type female insects.


SEQUENCE LISTING (7 sequences)

SEQ ID NO: 1 - DNA, 35 bp, Artificial Sequence
  Primer
gcaatgataccgcgggatccacgctcaccggctcc

SEQ ID NO: 2 - DNA, 35 bp, Artificial Sequence
  Primer
ggagccggtgagcgtggatcccgcggtatcattgc

SEQ ID NO: 3 - DNA, 33 bp, Artificial Sequence
  Primer
gccaggacgtcctttgtatgcgtgcgcttgaag

SEQ ID NO: 4 - DNA, 22 bp, Artificial Sequence
  Primer
ccgtataagttcgagatcggcc

SEQ ID NO: 5 - DNA, 22 bp, Artificial Sequence
  Primer
cacatcacatcacaaggagtgc

SEQ ID NO: 6 - DNA, 22 bp, Artificial Sequence
  Primer
taggaaaggttagaaaagcgac

SEQ ID NO: 7 - DNA, 19 bp, Artificial Sequence
  Primer
ccgtattggccacgtgtcc